(19) United States
(12) Patent Application Publication
(10) Pub. No.: US 2012/0156634 A1
(43) Pub. Date: Jun. 21, 2012

(54) Intraoral Imaging Devices And Methods

(75) Inventors: Ronald Richard Duff, JR., Shrewsbury, MA (US); Thomas Aurele Christman, Greenwood, VA (US); Marianne Zsiska, Middletown, OH (US); Richard H. Cohen, Sherborn, MA (US); Mark David Vallon, Framingham, MA (US)

(73) Assignee: The Procter & Gamble Company, Cincinnati, OH (US)

(21) Appl. No.: 12/974,215

(22) Filed: Dec. 21, 2010

(51) Int. Cl. A61B 1/24

(52) U.S. Cl. 433/29; 433/215

(57) ABSTRACT

Disclosed is an intraoral imaging method that includes providing an intraoral imaging device having at least one biteplate including a dental impression material and a track, at least one carriage assembly that travels along at least a portion of the track, and imaging equipment which is secured to the at least one carriage assembly; locating the at least one biteplate between upper and lower teeth of the subject such that the dental impression material cooperates with at least one of the upper and lower teeth to provide repeatable registration within an oral cavity of the subject; and obtaining image data using the imaging equipment.

### TECHNICAL FIELD

The present disclosure relates to devices and methods utilized for intraoral imaging.

### BACKGROUND

Current methods of measuring clinical performance of plaque removal often involve subjective assessments. Likewise, current methods of measuring other dental standards that relate to the health and/or cosmetic appearance of teeth and gums also involve subjective assessments. Such subjective assessments naturally include a degree of variability. More uniform and objective methods of measurement are of continued interest.

### SUMMARY

One embodiment of an intraoral imaging method includes providing an intraoral imaging device having at least one biteplate including a dental impression material and a track, at least one carriage assembly that travels along at least a portion of the track, and imaging equipment which is secured to the at least one carriage assembly; locating the at least one biteplate between upper and lower teeth of the subject such that the dental impression material cooperates with at least one of the upper and lower teeth to provide repeatable registration within an oral cavity of the subject; and obtaining image data using the imaging equipment.

Another embodiment of an intraoral imaging method includes providing an intraoral imaging device having a biteplate including a dental impression material and a track, a carriage assembly that travels along at least a portion of the track, and imaging equipment which is secured to the carriage assembly and in communication with computing equipment; locating the biteplate between upper and lower teeth of the subject such that the dental impression material cooperates with at least one of the upper and lower teeth to provide repeatable registration within an oral cavity of the subject; obtaining image data using the imaging equipment; sending the image data from the imaging equipment to the computing equipment; and utilizing the computing equipment to generate a plurality of images of at least one of the upper and lower teeth of the subject based on the image data received from the imaging equipment.

One embodiment of an intraoral imaging device includes at least one biteplate having a dental impression material and a track; at least one carriage assembly that travels along at least a portion of the track; and imaging equipment which is secured to the carriage assembly and in communication with computing equipment, wherein the imaging equipment includes a viewing tip that includes a camera lens, an illumination lens, and at least one window covering the camera and illumination lenses that includes at least one polarizing filter.

### BRIEF DESCRIPTION OF THE DRAWINGS

FIG. 1 depicts an embodiment of an intraoral imaging device;

FIG. 2 depicts a portion of the embodiment of the intraoral imaging device of FIG. 1;

FIG. 3 depicts a portion of the imaging equipment of the embodiment of the intraoral imaging device of FIG. 1;

FIG. 4 depicts the viewing tip of the imaging equipment of the embodiment of the intraoral imaging device of FIG. 1;

FIG. 5 depicts an embodiment of a probe cap; and

FIG. 6 schematically depicts the steps of an embodiment of an intraoral imaging method.

### DETAILED DESCRIPTION

As will be discussed herein, embodiments of an intraoral imaging device 100 may comprise a biteplate 110 including a dental impression material 112 and a track 120, a carriage assembly 130, imaging equipment 140, a light source 150 and computing equipment 160. It should be understood, however, that embodiments of intraoral imaging device 100 may also include further structure, such as, for example, a disposable probe cap 170.

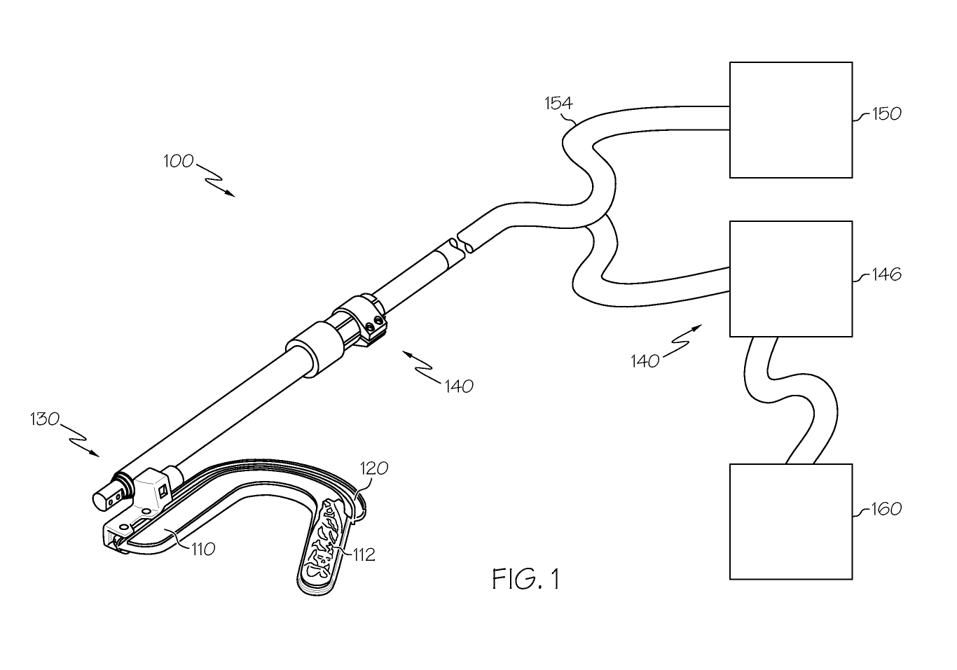

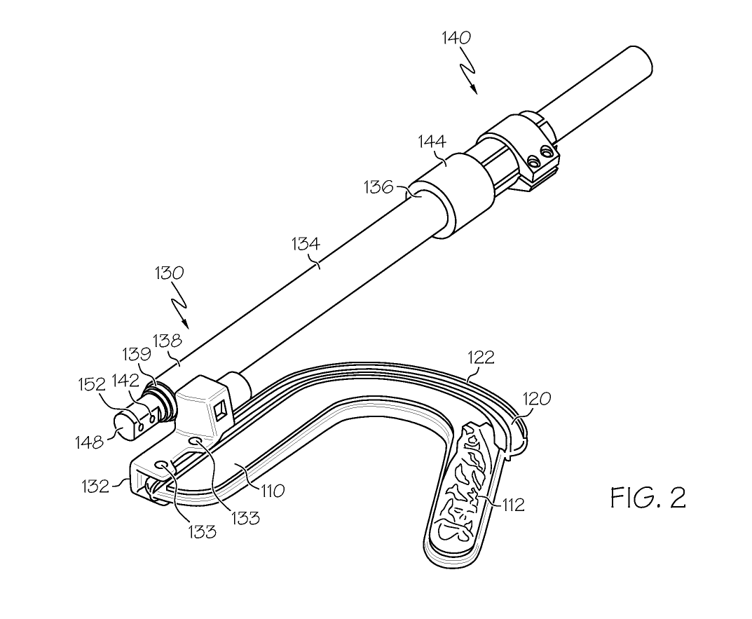

Referring to FIGS. 1 and 2, embodiments of intraoral imaging device 100 may include a biteplate 110, which may be constructed of any material known in the art. In some embodiments, biteplate 110 may be constructed of plastic, and more specifically, hard engineering thermoplastic. In one non-limiting embodiment, biteplate 110 may be constructed of DuPont Delrin 500P NC010 with blue ECM POM343® colorant. A biteplate that utilizes blue ECM POM343® colorant may be useful in intraoral imaging methods where an embodiment of intraoral imaging device 100 is employed to image plaque that has been highlighted with a red erythrosine stain. Because blue colorant has a very low red channel component, the color of the biteplate employing blue ECM POM343® colorant will not conflict with the detection and measurement of red-stained plaque. However, different plastics and/or colorants may be utilized in the construction of biteplate 110 depending on the particular application of intraoral imaging device 100. Moreover, other suitable stains may be utilized in embodiments of plaque imaging methods, including, but not limited to, fluorescein, and methylene blue. Additionally, biteplate 110 may be formed through any method known in the art. Non-limiting examples include injection molding, thermoforming, blow molding, compression molding, and rotational molding.

Biteplate 110 may be constructed in multiple sizes for use with different subjects. Biteplate 110 may include a dental impression material 112 disposed along at least a portion of the biteplate to create a custom fit and registration for the teeth of a particular subject. Accordingly, when biteplate 110 is located between the upper and lower teeth of a subject, dental impression material 112 may cooperate with at least one of the upper and lower teeth to provide a repeatable registration within the oral cavity of the particular subject (i.e., the biteplate may be repeatably positioned in the same location between the teeth of a particular subject). To cooperate with the upper and/or lower teeth of a subject, impression material 112 may be molded to form a negative of the profile of one or more teeth of the subject. Once molded, each time dental impression material 112 and a portion of the upper and/or lower teeth of a subject are brought together, specific protrusions and cavities in the dental impression material may cooperate (e.g., interface or match-up) with specific protrusions and cavities in the profile of the teeth. For intraoral imaging methods that include the obtaining of image data in multiple steps, the repeatable registration imparted by the cooperation between impression material 112 and at least one of the upper and/or lower teeth of a subject may provide for more accurate and/or precise measurements.

Although any impression material known in the art may be employed, one exemplary impression material is Exafast Fast Set Vinyl Polysiloxane™, Standard Package, P/N 137307. As depicted in FIGS. 1 and 2, impression material 112 may be disposed along at least a portion of the top surface of biteplate 110. In other embodiments, the impression material may be disposed along at least a portion of the bottom surface of biteplate 110. In yet other embodiments, the impression material may be disposed along at least a portion of both the top and bottom surfaces of biteplate 110. Biteplate 110 may further include a textured surface to aid in retaining impression material 112 on the biteplate.

Embodiments of intraoral imaging device 100 may include at least one biteplate 110 for each patient. In some embodiments of intraoral imaging device 100, two or four biteplates 110 may be utilized for each subject. In embodiments that include two biteplates 110, a first biteplate may be utilized for the imaging of at least one-half of the upper and/or lower teeth of a subject and a second biteplate may be utilized for the imaging of at least the other-half of the upper and/or lower teeth of the subject. One embodiment includes a first biteplate that may include dental impression material 112 on at least a portion of the left side of the biteplate so that the right side of the teeth of the subject can be imaged. A second biteplate may include dental impression material 112 on at least a portion of the right side of the biteplate so that the left side of the teeth of the subject can be imaged. This dual biteplate arrangement may assist in the imaging of the full set of upper and/or lower teeth of a patient without obstruction from dental impression material 112. Moreover, a first biteplate which includes dental impression material 112 on at least a portion of the right side may cooperate with a first carriage assembly (described in detail below) to image the upper left teeth of the subject and may cooperate with a second carriage assembly to image the lower left teeth of the subject. Similarly, a second biteplate which includes dental impression material 112 on at least a portion of the left side may cooperate with each of the carriage assemblies to image the remaining upper and lower right quadrants. In embodiments that include four biteplates 110, a separate biteplate may be created for imaging each of at least a first half of the upper teeth, at least a second half of the upper teeth, at least a first half of the lower teeth and at least a second half of the lower teeth of a subject.

Still referring to FIGS. 1 and 2, intraoral imaging device 100 may include a track 120 disposed along at least a portion of the outside perimeter of biteplate 110 that cooperates with a carriage assembly 130 (described in further detail below). Track 120 may include guide rail 122 that engages with structure (such as pins 133) disposed on carriage assembly 130. The cooperation between carriage assembly 130 and guide rail 122 facilitate the carriage assembly, and thus the imaging equipment secured to the carriage assembly, in traveling along the outside perimeter of biteplate 110. While traveling along track 120, carriage assembly 130 may be able to stop at random or predetermined locations. Moreover, some embodiments of intraoral imaging device 100 may further include structure on track 120 and/or biteplate 110 that assists carriage assembly 130 in stopping at predetermined locations to aid imaging equipment 140 in obtaining image data from a number of static positions. The static position of imaging equipment 140 during the imaging process may help eliminate motion blur which is sometimes associated with dynamic imaging. Non-limiting examples of structure may include a series of detents at predetermined positions along track 120 and/or biteplate 110. In such an embodiment, carriage assembly 130 may be configured with reciprocal structure, such as a ball plunger, for cooperation with the detents. Moreover, any structure known in the art may assist carriage assembly 130 in stopping at predetermined locations along track 120. However, such structure need not be included on embodiments of intraoral imaging device 100.

Additional structure may also be included on intraoral imaging device 100 to assist in the tracking and/or positioning of carriage assembly 130 along track 120 during the imaging process. In utilizing intraoral imaging device 100 for a particular imaging application, carriage assembly 130 may or may not stop at predetermined locations along track 120. Therefore, the following embodiments may be employed in applications utilizing static imaging and/or dynamic imaging. One embodiment of intraoral imaging device 100 may include a series of optically detectable features (e.g., bar coding or existing texture) on biteplate 110 and/or track 120 and an optical detector on carriage assembly 130. As carriage assembly 130 travels along track 120 during the imaging process, the optical detector identifies the optically detectable features on biteplate 110 and/or track 120 and provides tracking and/or positioning information to imaging equipment 140 and/or computing equipment 160. Another embodiment of intraoral imaging device 100 may include a series of magnetically detectable features (e.g., embedded magnets) on biteplate 110 and/or track 120 and a magnetic detector on carriage assembly 130. As carriage assembly 130 travels along track 120 during the imaging process, the magnetic detector identifies the magnetically detectable features on biteplate 110 and/or track 120 and provides tracking and/or positioning information to imaging equipment 140 and/or computing equipment 160. Yet another embodiment of intraoral imaging device 100 may include protrusions on biteplate 110 and/or track 120 and a rotatable gear on carriage assembly 130 that engages the protrusions and turns as the carriage assembly travels along the track. The rotatable gear spins an encoder (e.g. optical, magnetic or Hall effect) thus providing tracking and/or positioning information to imaging equipment 140 and/or computing equipment 160 during imaging. In such embodiments of intraoral imaging device 100, the rotating gear on carriage assembly 130 may further be driven by a miniature motor which may propel the carriage assembly along track 120 at a selected and/or constant velocity. Movement of carriage assembly 130 at a constant velocity reduces or eliminates accelerations and/or decelerations caused by the manual positioning of the carriage assembly. In embodiments of intraoral imaging device 100 that utilize dynamic imaging, the reduction or elimination of accelerations and/or decelerations will reduce or eliminate motion blur and assist in obtaining more accurate image data.

As illustrated in FIGS. 1 and 2, carriage assembly 130 may comprise a carriage portion 132 and a sheath portion 134 and may be utilized to accurately align imaging equipment 140 with biteplate 110. Carriage portion 132 may comprise any structure that cooperates with and travels along track 120 disposed on the perimeter of biteplate 110. In one embodiment, to maintain the proper positioning of imaging equipment 140 while obtaining image data, the clearance between guide rail 122 and the cooperating structure of carriage portion 132 may be reduced as much as possible to minimize play while still allowing the carriage portion to smoothly travel along the guide rail. As mentioned above, the cooperating structure of carriage portion 132 may include, for example, pins 133 to assist the carriage portion in engagement with guide rail 122. In some embodiments, carriage portion 132 may only be able to engage and disengage with guide rail 122 at the distal ends of the guide rail. Accordingly, carriage portion 132 may remain engaged with guide rail 122 as the carriage assembly 130 travels around track 120.

Sheath portion 134 may be a hollow tube constructed of stainless steel, various plastics, including, but not limited to, DuPont Delrin®, and/or any other material known in the art. In embodiments of intraoral imaging device 100 that include imaging equipment 140 with a flexible neck (e.g., the flexible braided cable of a videoscope), sheath portion 134 may function to stiffen the flexible neck and therefore assist in maintaining the proper positioning of the imaging equipment. Imaging equipment 140 may be inserted into a first end 136 of sheath portion 134 with the viewing tip 148 exiting at a second end 138 of sheath portion 134. Imaging equipment 140 may further be fitted with a custom collar 144 that interfaces with first end 136 of sheath portion 134 and operates to maintain the engagement of the sheath portion with the imaging equipment.

Carriage portion 132 may connect to sheath portion 134 through any method known in the art. Non-limiting examples include welding and soldering, as well as the use of adhesive, fasteners and/or engaging structure, such as, for example, structure for a slip fit arrangement. Carriage portion 132 and sheath portion 134 may also be formed in a one-piece construction of carriage assembly 130. The relative positioning of carriage portion 132 to sheath portion 134 may be selected to ensure the optimum spacing between camera lens 142 of imaging equipment 140 and the tooth and/or gum surface of the subject for the most accurate imaging. As detailed above, in some embodiments of intraoral imaging device 100, a set of mirror image carriage assemblies 130 may assist in imaging all four quadrants of the teeth of a subject. A first carriage assembly may be used to acquire the upper left and lower right quadrants of the teeth and a second carriage assembly (a mirror of the first carriage assembly) may be used to acquire the lower left and upper right quadrants of the teeth. Carriage assembly 130 may be disposable or reusable. In embodiments of intraoral imaging device 100 that include reusable carriage assembly 130, the carriage assembly can be sterilized by autoclave or cold sterilization solutions such as Sporox® and/or glutaraldehyde.

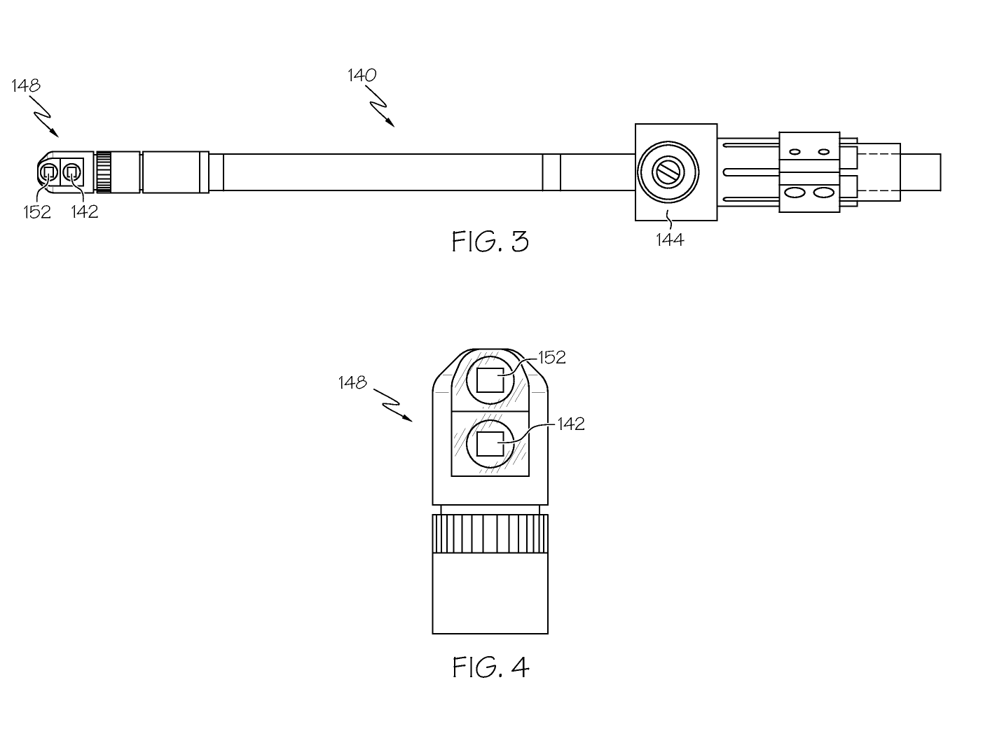

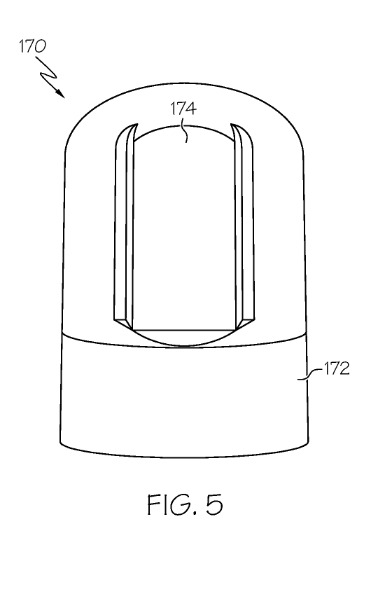

Referring to FIGS. 1-4, the intraoral imaging device may include imaging equipment 140 for obtaining image data of the teeth of a subject. As used herein, “obtaining image data” is defined as including, but not limited to, capturing, saving and/or transmitting image data. Any imaging equipment 140 may be utilized in intraoral imaging device 100, but some non-limiting options include videoscopes, borescopes, fiberscopes and intraoral cameras. One exemplary device is the Olympus® IV6C6-13 (6 mm diameter) videoscope (depicted in FIGS. 3 and 4). In some embodiments, imaging equipment 140 may also comprise a camera control unit 146, such as the Olympus® IV-6A, which provides dual NTSC S-Video outputs. A light source 150 may also be utilized to illuminate the teeth of a subject. Any light source (e.g., white light, UV, IR, Near Infrared, etc.) may be utilized in intraoral imaging device 100, but a non-limiting option includes a 300 watt Xenon light source that employs a light guide 154 to deliver light to the illumination lens 152 at the viewing tip 148. Exemplary light sources include the Olympus® ILV-2 and Luxtec® 9300 models.

Imaging equipment 140 may include any suitable viewing tip 148. In embodiments of intraoral imaging device 100 that employ the Olympus® IV6C6-13 videoscope, the Olympus® IV6C6-AT120S/FF Far Focus Side View Tip is one non-limiting option for viewing tip 148. Such a videoscope tip is a side viewing tip adaptor with a 120° field of view and 7 mm-90 mm depth of field. Referring to FIGS. 3 and 4, viewing tip 148 may include at least one window that covers and protects a camera lens 142 of imaging equipment 140 and/or an illumination lens 152 of light source 150. In an effort to eliminate, or at least significantly reduce, glare from the specular reflection of light emanating from illumination lens 152, the window(s) of viewing tip 148 may be polarized. Any suitable circular and/or linear polarizing method may be employed. The glass of the window(s) may be directly polarized, or at least one polarizing filter may be employed to fit over the window(s) that covers and protects illumination lens 152 and camera lens 142. One non-limiting option is a filter created from Edmund Optics TECHSPEC® Linear Polarizing Laminated Film (P/N NT43-781). If a laminated film is used, the laminated film may be CNC (“computer numerically controlled”) machined and applied in a crossed polarized arrangement to form a custom filter which will maximize reduction of specular reflection (i.e., the linear polarizing film applied over the portion of the window(s) covering illumination lens 152 is configured perpendicular to the linear polarizing film applied over the portion of the window(s) covering camera lens 142). The polarizer(s) may be potted on the viewing tip window(s) using an optically clear potting compound. Any suitable optically clear epoxy or adhesive may be utilized to pot the polarizer(s) on the window(s) of viewing tip 148. One non-limiting example of such an optically clear epoxy is Loctite Hysol® E-00CL, P/N 29289.

The polarizing of the window(s) of viewing tip 148 may substantially reduce the reflected glare resulting in more stable camera gain control, as well as no loss of measurable tooth surface area. However, in some embodiments of intraoral imaging device 100, the window(s) of viewing tip 148 may not utilize polarization. Embodiments of intraoral imaging device 100 not utilizing polarization on the window(s) of viewing tip 148 may be utilized in intraoral imaging methods for gloss/shine measurements.

Intraoral imaging device 100 may include a probe cap 170 (as illustrated in FIG. 5) to provide an optically clear casement to cover and protect viewing tip 148. Probe cap 170 may be injection molded from low birefringence plastic in order to maintain the crossed polarization glare reduction performance of a customized viewing tip as described above. Any low birefringence plastic may be employed, but one non-limiting option is Arkema Altuglas Plexiglas® VS-100. Probe cap 170 may be secured to sheath portion 134 of carriage assembly 130 by any method known in the art. As illustrated in FIGS. 2 and 5, second end 138 of sheath portion 134 may include at least one machined step 139 utilized for engaging a mounting portion 172 of probe cap 170. Accordingly, a friction fit engagement between mounting portion 172 and machined step(s) 139 may secure probe cap 170 to second end 138 of sheath portion 134, ensuring a liquid tight seal to prevent contamination of viewing tip 148 and other components of imaging equipment 140 from liquid/saliva in the mouth of a subject.

Probe cap 170 may comprise a flat section 174 oriented over the window(s) of viewing tip 148 that may assist in the obtaining of accurate image data by imaging equipment 140 (e.g., reduced glare due to the flat shape). Embodiments of probe cap 170 may further include means, such as a cutout (not shown) on mounting portion 172 that cooperates with a protrusion in sheath portion 134 in order to reduce or prevent rotation of the probe cap and maintain the proper orientation of flat section 174 (i.e., over the window(s) of viewing tip 148). Probe cap 170 may be disposable or reusable. In embodiments of intraoral imaging device 100 that include a reusable probe cap, probe cap 170 can be sterilized by autoclave or cold sterilization solutions such as Sporox® and/or glutaraldehyde.

Referring to FIG. 1, intraoral imaging device 100 may include computing equipment 160 that cooperates with imaging equipment 140. The image data obtained by imaging equipment 140 may be sent to computing equipment 160 by any method known in the art, including through the utilization of wires or wirelessly. The image data may be digital, analog, or in any other format, now or hereinafter known in the art. Moreover, data acquisition and/or digitization may be achieved by any computing equipment known in the art. One non-limiting option is an IBM®-compatible PC, such as a Dell® PC with dual Xeon® processors running Windows XP Pro, configured with video capture hardware and software such as Digital Rapids Stream 500 PCI hardware and Stream LE® software. In such an embodiment, the capture hardware digitizes an analog S-Video video stream from imaging equipment 140 and creates an audio video interleave (“AVI”) file. Color processing may be carried out in real time during the digitization process in order to optimize the image data for downstream analysis.

The embodiments of intraoral imaging device 100 described above may be utilized in various intraoral imaging applications and methods. One such application is the measuring of plaque levels on the teeth of a subject. Other applications for intraoral imaging device 100 may include measuring stain on teeth, detecting cavities/caries on or within teeth, measuring the whiteness of teeth, measuring the cleanliness of teeth, measuring the gloss/shine of teeth, measuring gum recession, measuring gingivitis indicators, and measuring any other dental standard that relates to the health and/or cosmetic appearance of teeth, gums, and/or other oral cavity tissues. Embodiments of the intraoral imaging devices for the above described applications may or may not employ some or all of the particular elements detailed above. For example, embodiments of intraoral imaging devices employed in applications to measure the gloss/shine of teeth may lack polarizing filters on the window(s) of the viewing tip.

In some embodiments, the intraoral imaging devices described herein may also be utilized to image teeth and/or gums as well as identify problem areas and/or perform a process upon teeth and/or gums. As a non-limiting example, embodiments of intraoral imaging devices may be employed to image and detect areas containing plaque in the oral cavity of a user and then spot treat such areas. In some embodiments, the equipment utilized in identifying problem areas and/or spot treating such areas may be separate from the intraoral imaging devices described above. In other embodiments, such equipment may be incorporated within the intraoral imaging devices.

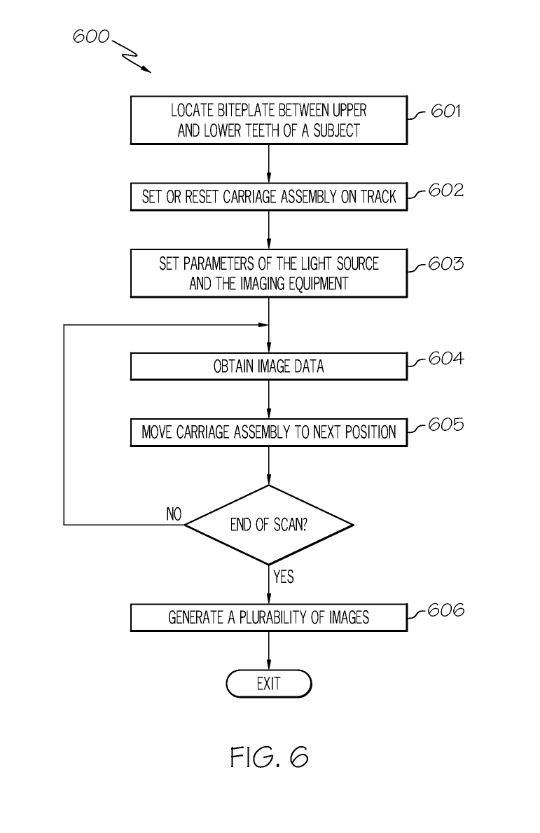

Referring to FIG. 6, an exemplary intraoral imaging method 600 is schematically depicted. In a first step, biteplate 110 is located between the upper and lower teeth of a subject (step 601). In this step, dental impression material 112 cooperates with at least one of the upper and lower teeth to provide a repeatable registration within the oral cavity of the subject. In a second step, carriage assembly 130 may then set or reset to an initial position along track 120 before beginning the imaging process (step 602). In a third step, the parameters of light source 150 and imaging equipment 140 may be set (step 603). Imaging equipment 140 may then obtain (e.g., capture, save, transmit, etc.) image data of the teeth of a subject (step 604). In embodiments of methods that include transmitting image data, the data may be sent to computing equipment 160 that is in communication with imaging equipment 140. Carriage assembly 130 may then automatically or manually be moved to a next position on track 120 (step 605). At each predetermined position, imagining equipment 140 may obtain image data of the teeth of a subject. Once imaging equipment 140 has obtained the desired amount of image data, computing equipment (internal to the imaging equipment or separate from the imaging equipment) may generate a plurality of images of at least one of the upper and lower teeth of the subject based on the image data received from the imagining equipment (step 606).

In some embodiments of intraoral imaging methods, not all of the above detailed exemplary steps are necessary. As an example, some embodiments of intraoral imaging methods only include steps 601-605. Moreover, additional steps may be included in some embodiments of intraoral imaging methods that were not detailed above. As an example, in embodiments of intraoral imaging methods for detecting plaque on the teeth of a subject, an initial step of applying a stain to at least one of the upper and lower teeth of a subject may be employed before locating the biteplate between the upper and lower teeth of a subject. Accordingly, embodiments of intraoral imaging methods may include steps that incorporate, employ or utilize any structure or function of the intraoral imaging devices detailed herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An intraoral imaging method comprising:
providing an intraoral imaging device comprising at least one biteplate including a dental impression material and a track, at least one carriage assembly that travels along at least a portion of the track, and imaging equipment which is secured to the at least one carriage assembly;
locating the at least one biteplate between upper and lower teeth of the subject such that the dental impression material cooperates with at least one of the upper and lower teeth to provide repeatable registration within an oral cavity of the subject; and
obtaining image data using the imaging equipment.

2. The method of claim 1, further comprising the step of applying a stain to at least one of the upper and lower teeth of a subject.

3. The method of claim 1, wherein the imaging equipment is in communication with computing equipment.

4. The method of claim 3, further comprising the steps of sending the image data from the imaging equipment to the computing equipment, and utilizing the computing equipment to generate a plurality of images of at least one of the upper and lower teeth of the subject based on the image data received from the imaging equipment.

5. The method of claim 4, wherein the plurality of images are generated as a sequence representing a video of at least one of the upper and lower teeth of the subject.

6. The method of claim 1, wherein the track includes a plurality of predetermined locations where the imaging equipment is located during the obtaining of the image data.

7. The method of claim 6, wherein the predetermined locations are disposed at positions on the track adjacent to detents disposed on at least one of the at least one biteplate and the track.

8. The method of claim 6, wherein the predetermined locations are disposed at positions on the track adjacent to optically detectable features disposed on at least one of the at least one biteplate and the track.

9. The method of claim 6, wherein the predetermined locations are disposed at positions on the track adjacent to magnetically detectable features disposed on at least one of the at least one biteplate and the track.

10. The method of claim 1, wherein the intraoral imaging device further comprises a plurality of protrusions disposed on the track and a rotatable gear disposed on the at least one carriage assembly.

11. The method of claim 10, wherein the intraoral imaging device further comprises a motor to drive the rotatable gear of the at least one carriage assembly along the protrusions of the track.

12. An intraoral imaging method comprising:
providing an intraoral imaging device comprising a biteplate including a dental impression material and a track, a carriage assembly that travels along at least a portion of the track, and imaging equipment which is secured to the carriage assembly and in communication with computing equipment;
locating the biteplate between upper and lower teeth of the subject such that the dental impression material cooperates with at least one of the upper and lower teeth to provide repeatable registration within an oral cavity of the subject;
obtaining image data using the imaging equipment;
sending the image data from the imaging equipment to the computing equipment; and
utilizing the computing equipment to generate a plurality of images of at least one of the upper and lower teeth of the subject based on the image data received from the imaging equipment.

13. The method of claim 12, wherein the plurality of images are generated as a sequence representing a video of at least one of the upper and lower teeth of the subject.

14. The method of claim 12, wherein the intraoral imaging device further comprises a second biteplate.

15. The method of claim 14, wherein the intraoral imaging device further comprises a third biteplate and a fourth biteplate.

16. An intraoral imaging device comprising:
at least one biteplate including a dental impression material and a track;
at least one carriage assembly that travels along at least a portion of the track; and
imaging equipment which is secured to the carriage assembly and in communication with computing equipment, wherein the imaging equipment includes a viewing tip that includes a camera lens, an illumination lens, and at least one window covering the camera and illumination lenses that includes at least one polarizing filter.

17. The device of claim 16, wherein the at least one polarizing filter comprises a first linear polarizing film.

18. The device of claim 17, wherein the first linear polarizing film is disposed on a portion of the window covering the camera lens and a second linear polarizing film is disposed on a portion of the window covering the illumination lens, wherein the first and second polarizing films are configured to be potted in a cross polarized arrangement.

19. The device of claim 18, wherein the first linear polarizing film and the second linear polarizing film are potted using a clear potting compound.

20. The device of claim 16, further comprising a probe cap constructed of a low birefringence plastic disposed over the viewing tip.
